(19) United States
(12) Patent Application Publication
(10) Pub. No.: US 2021/0330301 A1
(43) Pub. Date: Oct. 28, 2021

(54) DEVICE FOR COLLECTING POLYPS

(71) Applicant: A.C. HOOGERHEIDE HOLDING B.V., Eindhove (NL)

(72) Inventor: Adriaan Cornelis Hoogerheide, Volendam (NL)

(21) Appl. No.: 17/282,226

(22) PCT Filed: Oct. 3, 2019
(86) PCT No.: PCT/IB2019/001120

§ 371 Date: Apr. 1, 2021

(30) Foreign Application Priority Data
Oct. 4, 2018 (NL) NL2021759

(51) Int. Cl. A61B 10/00; A61B 10/04

(52) U.S. Cl. CPC A61B 10/0096; A61B 10/04

(57) ABSTRACT

The invention pertains to a device for catching a polyp, comprising a housing with a holder for catching the polyp, a supply connection for coupling with a supply tube and a drain connection for coupling with a drain pipe, where the housing and the holder are fitted to place the holder at a position between the supply connection and the drain connection for placing the polyp in a reception area of the holder. The device is characterized by the fact that the housing has a longitudinal passage cavity with an input opening for placing the holder in the main cavity and an output opening for removing the holder from the cavity. The invention also relates to an assembly of a housing and at least two holders and also a method of catching a polyp.

The present invention relates to a device for collecting polyps according to the introductory phrase of claim 1. The invention also relates to an assembly of a housing and a holder. The invention also relates to a method of catching a polyp.

Such a device is known in the state of art. These are used to collect or catch polyps and the like which are removed from a patient's body via an endoscope. A pump provides the suction through the endoscope to the device by connecting a drain pipe between the device and the pump.

The well-known device provides the possibility to catch polyps. Users believe that the use of the device to catch and transport the polyps, for example to research equipment, can be improved. Therefore, there is a need for an alternative device for collecting polyps and the like. In particular, there is a need for a device that can be used In an easy way without a multitude of parts required.

The invention is now intended to provide an improved design of the type mentioned in the introductory phrase.

In particular, the purpose of the invention is to provide a device of the type listed in the introductory phrase which ensures continuous use.

The invention also aims to provide an improved device that allows a user to quickly catch and transmit multiple polyps in transport cups for further analysis and treatment.

In order to obtain at least one of the aforementioned benefits, the invention shall provide a device which contains the measures of claim 1, in accordance with a first embodiment. This device has the advantage that a user can quickly catch a polyp, can transfer the polyp into a transport cup and then can catch a next polyp.

It has also been found that the device can be cleaned easily, thoroughly and quickly according to the invention, making it efficient to use by allowing a user to continuously work without having to use new parts continuously. Such a synergistic effect is a great advantage.

The invention therefore relates to a device for catching a polyp, comprising a housing with a holder for catching the polyp, a supply connection for coupling with a supply tube and a drain connection for coupling with a drain tube, where the housing and the holder are fitted to place the holder at a position between the supply connection and the drain connection for placing the polyp in a reception area of the holder, which device is characterized by the fact that the housing has a longitudinal passage cavity with an input opening for placing the holder in the main cavity and an output opening for removing the holder from the cavity. This provides an alternative to the in-practice device used to catch a polyp. This new device according to the invention works simply, quickly and efficiently.

It is preferable that the holder and the housing have mutually cooperating seals to prevent air supply from the environment to the reception area of the holder. As a result, a secure supply of the polyp is ensured by the supply tube and a total drain of liquid and gas through the drain pipe. Not only during the normal use of the device, but especially when a holder is removed from the housing, when a polyp is collected, it is preferable that the seal stays intact so that no false air from the environment is sucked into the drain tube.

A sealing can be obtained by applying a flexible material to at least part of the exterior of the device, for example on a position upstream of the reception area and downstream of the reception area, relative to the direction with which the holder is fed through the housing, which seals the reception area from the environment. It is therefore preferable that, according to the invention, the holder of the device comprises a flexible material.

In order to obtain a verification of the presence and condition of a polyp, it is preferable for the housing to comprise a transparent material at the position of the holder's reception area when positioning the holder between the supply connection and the drain connection. This allows a doctor or assistant to check whether treatment is going well.

An improved control can be obtained when the housing includes a magnifying lens at the position of the holder's reception area when positioning the holder between the supply connection and the drain connection for magnifying the content of the reception area.

The device can be used in an appropriate manner continuously. To this end, it is preferable that the housing has a sufficient length to substantially contain two holders. A first holder can then be placed in a position (the position of use) before use by placing it in the housing from housing inlet and sliding it to that position of use, where the reception area of that first holder is located between the supply connection and the drain connection. A second holder can be positioned in a downstream position in the housing. That second holder may be used to move the first holder to its position of use where the second holder pushes the first holder forward to that position of use. Optionally, the length of the housing may be shorter than necessary to fully accommodate two holders, but the length should preferably be such that the second holder is at least partially incorporated into the housing and remains positioned in it when a user who releases the second holder after the first holder has been placed in the position of use. It is more preferred that when the reception area of the second holder is enclosed by the housing so that no contamination can take place of the reception area of the second holder, indicated here by the expression that at least two holders are held by the housing. The invention therefore preferably relates to a device in which the housing is designed to hold at least two holders.

According to a preferred embodiment, the holder for application in the housing has a front and rear panel, and is configured to be placed in the housing with the front end first, whereby a protruding edge around a position at the front of the reception area and at a position at the rear end of the reception area for sealing the reception area from the environment when placing the holder in the housing. The protruding edge provides an airtight sealing of the reception area from the environment, so that no false air is sucked in and a continuous and efficient operation of the extraction is secured.

A precise and simple positioning is provided when the housing in the continuous cavity has at least one notch which, when meeting the protruding edge of the holder, provides a positioning indication. The notch and the protruding edge should be positioned in such a manner, that the reception area of the holder is located exactly at the position of the supply connection and drain connection. Two protruding edges and two notches may be provided, but a single notch is also effective, where only a single protruding edge is received in a notch.

In order to provide a suitable throughput of liquid and gas through the reception area and catching of the polyp, it is preferable that the holder includes a bottom with a perforation for liquid and gas to pass, and for solid materials, like a polyp, to be caught. The openings can have a dimension of about 1 mm diameter, but different sizes are also applicable within the scope of the invention.

To ensure at all times a sealing of the environment to the drain connection, it is preferable that the distance from the drain connection for coupling with a drain pipe to a rear of the housing, is greater than a distance between two positions on the holder which take care of the holder's seal in contact with the housing. As a result, when removed from the housing, the holder will not be able to pass air from the environment to the drain connection. When the sealing placed at the rear of the holder leaves the housing, the front sealing is located at a position between the end of the housing and the drain connection. Therefore, when the first holder is removed from the housing, the second holder takes the original position of the first holder.

According to another aspect, the invention relates to an assembly of a device according to the invention with a housing and at least two, preferably at least three holders.

According to another aspect, the invention relates to a method for catching a polyp, comprising the connection of a supply tube to the supply connection of the device according to the invention and a drain tube on the drain connection of the device, using a pump connected to the drain tube, suction of a mixture of liquid and a polyp and catching the polyp in the holder placed in the housing.

The invention will be further explained by the figures. The figures show:

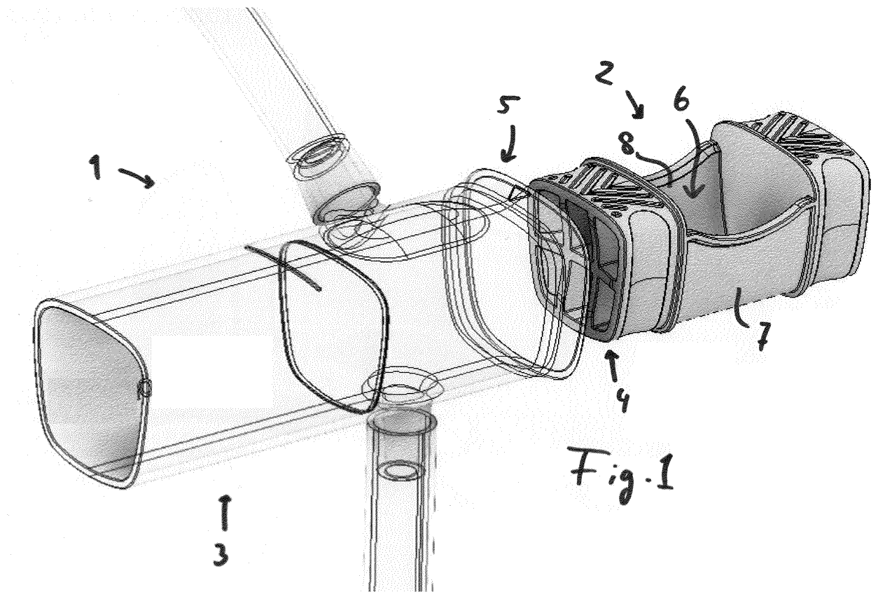

FIG. 1 a perspective view of the device with a housing and first holder,

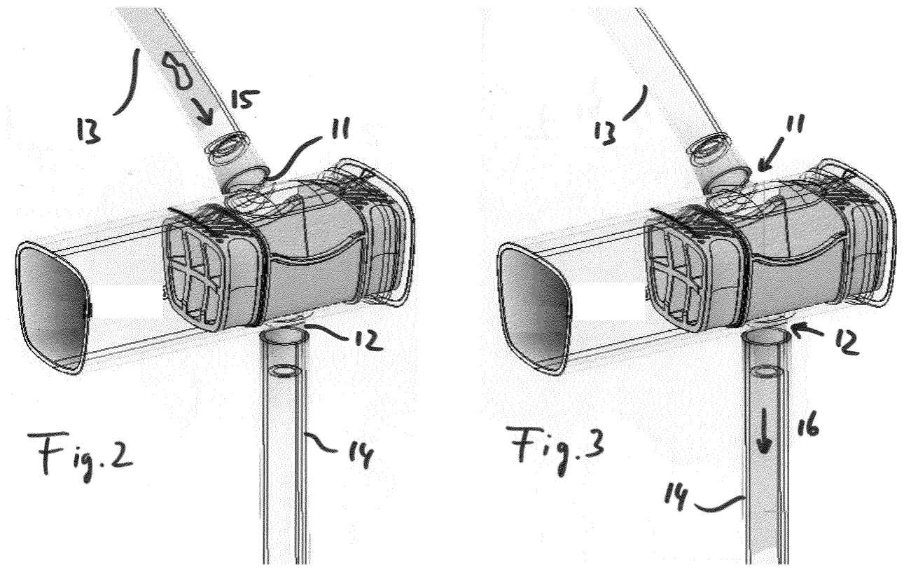

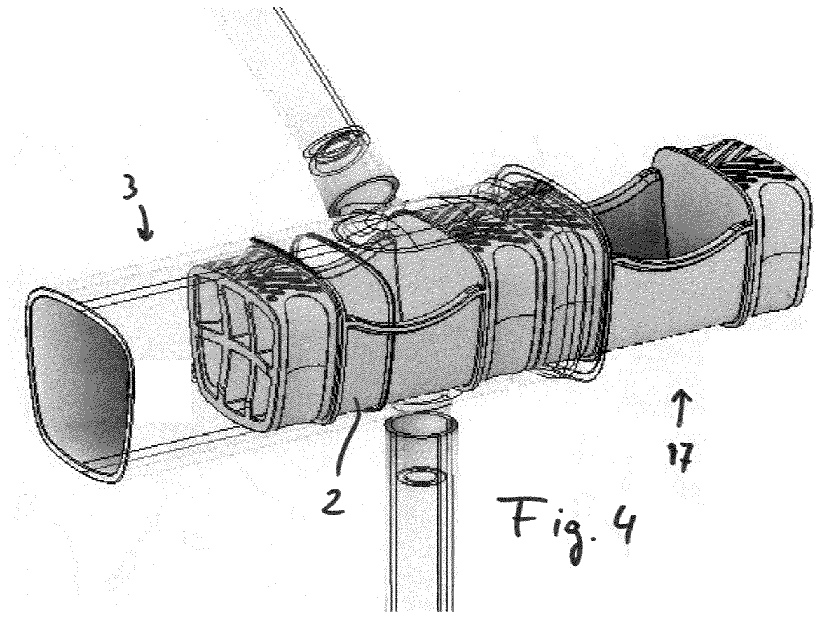

FIGS. 2 and 3 a perspective view of the device with a first holder in a position of use,

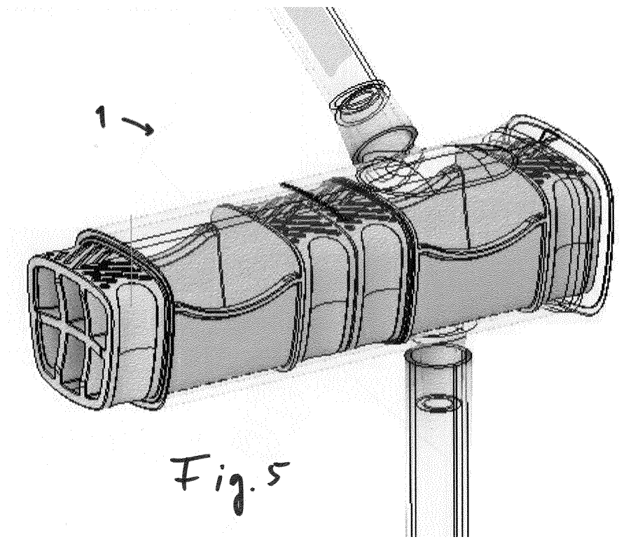

FIG. 4 a perspective view of the device with a first holder in a displaced position and a second holder,

FIG. 5 a perspective view of the device with a first and second holder, and

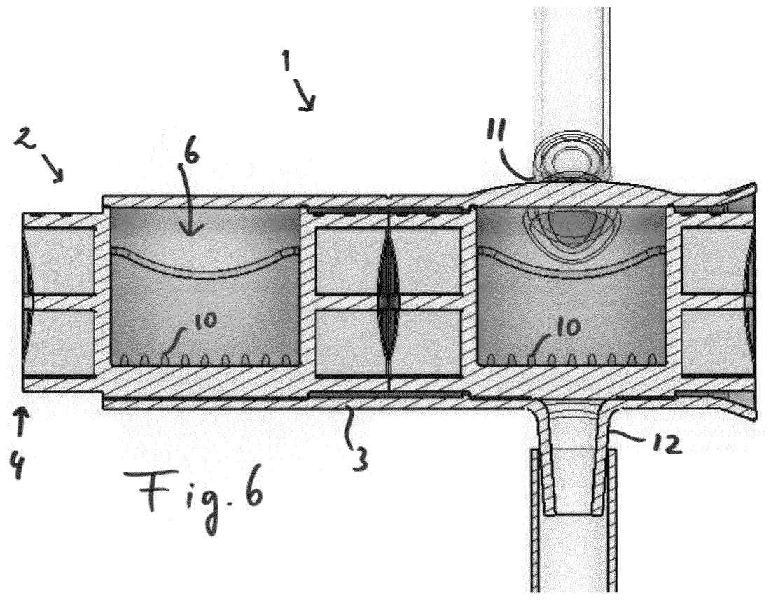

FIG. 6 A cross-section of the device according to FIG. 5.

The figures indicate the same parts with the same reference numbers. However, components necessary for a practical implementation of the invention are not all shown, because of the simplicity of the display.

FIG. 1 shows a Perspective view of a device 1 according to the invention with a first holder 2 placed at a position outside a housing 3. The holder 2 may be placed with a front 4 in a supply opening 5 of the housing 3. The holder has a reception area 6 for the catching of a polyp or similar. The reception area 6 has sidewalls 7, 8 and a bottom 9. The bottom 9 is provided with perforations 10, shown in FIG. 6, to allow liquid and gas to pass. The reception area is open on side opposing the bottom side. FIG. 2 shows a device 1 according to the invention in which the holder 2 is placed in the housing 3. The holder 2 is located in a first position or position of use. The reception area 6 is located between a supply connection 11 and a drain connection 12. The supply tube 11 is coupled with a supply pipe 13 which is connected to an endoscope (not shown), while the drain connection 12 is coupled with a suction pump (not shown) via a drain tube 14. A polyp and moisture are fed to the reception area via the supply tube 13, indicated by the arrow 15 in FIG. 2. FIG. 3 shows how moisture is drained through the drain tube 14 in the direction of the arrow 16.

The figures clearly show that the position of the supply connection is not displaced exactly over 180° relative to the position of the drain connection. As a result, at a position diametrically compared to (or directly opposite of) the drain connection, a direct view from above the holder is obtained. The angle between the drain connection and the supply connection, relative to a cross-section of the housing, is preferably less than 160°, more preferably less than 150°, yet even more preferably less than 140°, and in particular less than 125°. The angle is also preferably more than 90°, more preferably more than 110°.

After a polyp is caught in the reception area of the holder 2, a second holder 17 is used to push the first holder 2 in the direction of the outlet side 18 of the housing 3, as shown in FIG. 4. When the second holder 17 is at the original position of the first holder 2, the first holder 2, as in the displayed embodiment, sticks out of the housing 3 with the front end, as shown in FIG. 5. The first holder 2 can be removed from the housing in a secure manner, after which the polyp can be disposed from the reception area and transferred into a transport—or analysis cup (not shown). The holder 2 can be cleaned and stored for later use.

In FIG. 6 a cross-section side view of the device 1 is shown. The first holder 2 is partly pushed out of the housing and the second holder 18 is placed in a position of use. The reception area of the second holder 18 is located between the supply connection 11 and the drain connection 12.

The supply connection 12 may be configured as connection element 12 which embraces supply tube 13, instead of the connection element 12 being inserted in the supply tube 13.

The invention is not limited to the execution forms shown above and in the figures. The invention is only limited by the attached claims.

The invention also extends over any combination of measures that are described independently of each other.

1-13. (canceled)

14. A device for catching a polyp, comprising a housing with a holder for catching the polyp, a supply connection for coupling with a supply tube and a drain connection for coupling with a drain pipe, where the housing and the holder are arranged for positioning the holder at a position between the supply connection and the drain connection for placing the polyp in a reception area of the holder, wherein the housing has a longitudinal passage cavity with an inlet for inserting the holder in the passage cavity and an outlet for removing the holder from the cavity.

15. The device according to claim 14, wherein the holder and the housing have mutually cooperating seals to prevent air supply from the environment to the reception area of the holder.

16. The device according to claim 14, wherein the holder comprises a flexible material.

17. The device according to claim 14, wherein the housing includes a transparent material at the position of the holder's reception area when positioning the holder between the supply connection and the drain connection.

18. The device according to claim 17, wherein the housing includes a magnifying lens at the position of the holder's reception area when positioning the holder between the supply connection and the drain connection for magnifying the contents of the reception area.

19. The device according to claim 14, wherein the housing is arranged for holding at least two holders.

20. The device according to claim 14, wherein the holder has a front end and a rear end, the holder designed to be inserted in the housing with the front end first, the reception area provided with a circumferential protruding edge at a position at the front end of the reception area, and at a position at the rear end of the reception area, for the sealing of the reception area from the environment when positioning the holder in the housing.

21. The device according to claim 20, wherein the housing in the passage cavity has a notch which, when meeting the protruding edge of the holder, provides a positioning indication.

22. The device according to claim 14, wherein the holder comprises a bottom with a perforation for allowing liquid and gas to pass, and for solid materials to be caught.

23. The device according to claim 22, wherein a distance from the drain connection for coupling with a drain tube to a rear end of the housing is greater than a distance between two positions at the holder which provides sealing of the holder when placing in the housing.

24. The device according to claim 14, wherein the angle between the drain connection and the supply connection, relative to a cross section of the housing, is less than 160°.

25. The device according to claim 24, wherein the angle between the drain connection and the supply connection, relative to a cross section of the housing, is less than 150°.

26. The device according to claim 25, wherein the angle between the drain connection and the supply connection, relative to a cross section of the housing, is less than 140°.

27. The device according to claim 26, wherein the angle between the drain connection and the supply connection, relative to a cross section of the housing, is less than 125°.

28. The device according to claim 24, wherein the angle between the drain connection and the supply connection, relative to a cross section of the housing, is more than 90°.

29. The device according to claim 28, wherein the angle between the drain connection and the supply connection, relative to a cross section of the housing, is more than 110°.

30. A method of assembling a device according to claim 14, comprising providing a housing and providing at least two holders.

31. The method of according to claim 30, comprising providing at least three holders.

32. A method for catching a polyp, comprising the steps of connecting a supply tube to the supply connector of the device according to claim 14, connecting a drain tube to the drain connection of the device, using a pump which is connected to the drain pipe for suction of a mixture of liquid and a polyp, and catching the polyp in the holder which is placed in the housing.
